(19) United States
(12) Patent Application Publication
(10) Pub. No.: US 2024/0131216 A1
(43) Pub. Date: Apr. 25, 2024

(54) HYDROGEL FOR TREATING PRESSURE ULCERS

(71) Applicant: PAUL HARTMANN AG, Heidenheim (DE)

(72) Inventors: Safinaz ÖLMEZ-NOSWORTHY, Neu-Ulm (DE); Maja KRÄMER, Herbrechtingen (DE); Axel ECKSTEIN, Heidenheim (DE)

(73) Assignee: PAUL HARTMANN AG, Heidenheim (DE)

(21) Appl. No.: 18/269,233

(22) PCT Filed: Dec. 22, 2021
(86) PCT No.: PCT/EP2021/087200

§ 371 Date: Jun. 21, 2023

(30) Foreign Application Priority Data
Dec. 23, 2020 (DE) 10 2020 134 827.4

(51) Int. Cl. A61L 15/26; A61L 15/60; C08L 75/04

(52) U.S. Cl. CPC A61L 15/26; A61L 15/60; C08L 75/04

(57) ABSTRACT

The present invention relates to the use of hydrogel in the treatment of pressure ulcers and/or in the prevention of the formation of pressure ulcers, particularly of pressure ulcers caused by devices or by medicinal products.

The present invention relates to the use of hydrogel in the treatment of pressure ulcers and/or prophylaxis of the development of pressure ulcers, in particular device-related or medical device-related pressure ulcers.

Decubitus ulcer (also known in short form as decubitus or pressure ulcer) is generally defined as a trophic disorder of tissues, in particularly the skin and/or subcutaneous tissue, that is caused by external pressure exerted over a prolonged period with compression of vessels and local ischemia.

Decubiti can thus develop when pressure is exerted on soft tissue, interrupting some or all of the blood flow to said tissue. Shearing forces arising from friction on specific skin areas also significantly contribute to the development of decubiti. Such shearing forces arise from movement of the skin on surfaces of other materials. When the shearing forces that occur cause an affected person's skin to shift against the tissues below the skin, blood vessels in said tissues are also compressed, restricting or interrupting the blood flow to said tissues. Decubitus may thus possibly lead to maceration and/or necrosis of the affected tissue or cause infections.

Decubitus can be divided into the following stages, which can, for example, merge into one another depending on the duration and/or intensity of the pressure exerted.
Stage I: This is a persistent, localized erythema that remains even when pressure is relieved. The redness is sharply defined and may be indurated or abnormally warm. The skin is still intact.
Stage II: Blistering and skin abrasion occur in this phase, resulting in partial loss of skin. There is damage to the epidermis right up to parts of the dermis. A superficial wound or a shallow ulcer is present in this phase.
Stage III: In this advanced stage, loss of all layers of the skin can already be observed. Furthermore, damage to the subcutaneous tissue and possibly necrosis that may extend up to the underlying muscle tissue can be observed. Experience has shown that the necrotic tissue must first be demarcated before the full extent of the tissue damage can be seen. Stage III decubitus presents clinically as an open, deep ulcer.
Stage IV: In this extremely critical stage, all layers of the skin are lost with extensive destruction, tissue necrosis, or damage to muscles, bones or supporting structures (tendons, joint capsules). Stage IV decubitus presents clinically as a large, open and deep ulcer.

Here, stage II, stage III and stage IV decubiti are known as chronic wounds.

It is known that decubiti can develop, inter alia, during bed confinement, especially on parts of the body at which the skin is in direct contact with a bone, but also, for example, under ill-fitting prostheses and excessively tight plaster casts. The areas at which decubiti commonly develop is the skin covering the sacral region, coccyx, heel or hip bones. Other areas at which decubiti may develop are elbows, knees, joints and shoulders. Especially patients whose mobility is severely restricted are at risk of developing decubiti, since their ability to relieve pressure through independent movement where areas are under pressure is limited. Customary measures for treating, for example, bedridden patients include pressure distribution by frequent change of position or by providing the place to lie with pressure-reducing mattresses or cushions.

Another specific type of decubitus which can be attributed to the inability to ensure sufficient pressure relief if necessary at areas under pressure is, however, also increasingly coming into focus. These are device-related pressure ulcers. Said device-related pressure ulcers (or pressure sores) are considered to be localized damage to the skin and possibly underlying tissue due to the pressure exerted by a medical or other device. As can be seen, for example, when using a protective or artificial respiration mask or protective goggles, there is no possibility during their use of reducing the pressure present on the skin under the contact points of the mask through movement/repositioning of the device, and this can lead to the development of the described pressure ulcers with prolonged use.

Even though the mechanisms leading to the development of decubiti are not yet fully known, it is believed that measures used for the treatment and prophylaxis of decubiti should be aimed at reducing the pressure and shearing forces acting on skin and tissue.

For example, the Journal of Wound Care, Consensus Document, Vol. 29, No. 2, February 2020, proposes to this end the use of a hydrocolloid-containing wound dressing that can be placed between the skin and the corresponding device. However, the wound dressing appears to still have room for improvement in respect of some properties.

Therefore, there continues to be a need for materials which can be used to overcome the disadvantages of the prior art. Here, the use thereof shall allow the treatment of device-related pressure ulcers and, in particular, effectively prevent the development of new decubiti or the worsening of existing decubiti.

Furthermore, a gentle treatment of decubiti shall be made possible, in which an optimal microclimate especially with regard to moisture shall be made possible for the affected area.

Moreover, simple handling, high acceptance and/or simple inspection of the affected area shall be ensured with or without prior absorption of water and/or wound fluid.

The present invention according to claim 1 solves at least one of the aforementioned problems.

Overall, it has been found that, unexpectedly, the use of a hydrogel having a water content of from 40% to 90%, in accordance with the invention, allows the treatment of device-related pressure ulcers and can, in particular, effectively prevent the development of new decubiti or the worsening of existing decubiti. Furthermore, an optimal microclimate for the treatment of pressure ulcers can be made possible at the affected area. Moreover, it has been found that simple handling of the hydrogel used can be ensured even after the absorption of water and/or wound fluid, which leads to high acceptance by the patient and/or simple inspection of the affected area by the treating person.

The subject of the present invention is a hydrogel for use in the treatment of pressure ulcers and/or prophylaxis of the development of pressure ulcers, wherein the hydrogel has a water content of from 40% to 90%.

In other words, the subject of the invention is the use of a hydrogel for treatment of pressure ulcers and/or prophylaxis of the development of pressure ulcers, wherein the hydrogel has a water content of from 40% to 90%.

Furthermore, the invention relates to the use of a hydrogel for production of a wound dressing for treatment or prophylaxis of the development of pressure ulcers, wherein the hydrogel has a water content of from 40% to 90%.

A further subject of the invention is a kit containing a hydrogel and a protective or artificial respiration mask or protective goggles, wherein the hydrogel has a water content of from 40% to 90%.

A decubitus or pressure ulcer can be defined as above and can develop in the manner likewise described above. A common type of pressure ulcer can occur during prolonged bed confinement and is then also accordingly referred to as a bedsore.

In a preferred embodiment of the invention, the pressure ulcers treated by the hydrogel used according to the invention are device-related pressure ulcers, in particular device-related pressure ulcers falling under stage I or stage II as described above. These device-related pressure ulcers can be caused by the pressure exerted on the skin surface by a medical or other device, thus resulting in localized damage thereto and possibly to underlying tissue. Thus, the hydrogel used according to the invention can be applied to the skin under the contact points of an artificial respiration and/or protective mask or protective goggles in order to prevent or at least advantageously reduce possible development of device-related pressure ulcers.

In an alternatively preferred embodiment of the invention, the pressure ulcers treated by the hydrogel used according to the invention are medical device-related pressure ulcers.

In the context of the invention, the term hydrogel refers to a finely dispersed system composed of at least one solid phase and one liquid phase. Said solid phase forms a spongy, three-dimensional matrix (network), the pores of which are at least partially filled by a liquid (lyogel), the liquid being water in the present case. The two phases preferably penetrate one another completely. As a result of water absorption, the three-dimensional network can increase its volume through swelling without losing structural cohesion.

The hydrogel used according to the invention has a water content of from 40% to 90%, preferably from 50% to 88%, even more preferably from 55% to 86%, in particular from 60% to 85%. The water content of the hydrogel is determined in the manner described in the experimental part.

In connection with the present invention, a water content is to be understood as meaning the water which can theoretically be released from the hydrogel.

It has been found that, surprisingly, the hydrogel used according to the invention can advantageously reduce pressure peaks despite its high water content (and a consequently soft consistency).

The hydrogel used according to invention can have a water releasing capacity. In other words, this means that the hydrogel used according to the invention can release moisture, in particular water, to the area to be treated. In a preferred embodiment, the hydrogel used according to the invention can release from 3 to 20 mg of moisture per square centimeter (cm²), preferably from 5 to 15 mg of moisture per square centimeter (cm²), in particular from 8 to 12 mg of moisture per square centimeter (cm²), within 24 hours, measured in accordance with TM 0000232. The liquid is preferably water. Preferably, the hydrogel used according to the invention can release 8 mg of moisture per square centimeter (cm²), 8.5 mg of moisture per square centimeter (cm²), 9.0 mg of moisture per square centimeter (cm²), 9.5 mg of moisture per square centimeter (cm²), 10.0 mg of moisture per square centimeter (cm²), 10.5 mg of moisture per square centimeter (cm²), 11.0 mg of moisture per square centimeter (cm²), 11.5 mg of moisture per square centimeter (cm²) or 12 mg of moisture per square centimeter (cm²) within 24 hours. The water releasing ability of the hydrogel is determined in the manner described in the experimental part.

It has been found that the water content and/or the water releasing capacity of the hydrogel used according to the invention can allow not only agreeable wearing comfort, but also gentle treatment of skin areas affected by device-related pressure ulcers through the optimal supply of moisture.

The hydrogel used according to the invention can preferably absorb substances released from the wound, in particular wound exudate, thereby making it possible to prevent or at least advantageously reduce renewed contact with the wound (recontamination). In a preferred embodiment, the hydrogel used according to the invention has an absorption capacity of from 1 to 10 g/g (hydrogel), preferably from 1.1 to 5 g/g (hydrogel), even more preferably from 1.2 to 3 g/g (hydrogel), within 24 hours. The absorption capacity of the hydrogel is determined in the manner described in the experimental part.

In a preferred embodiment, the hydrogel used according to the invention has a pH of from 5 to 9, preferably from 6.7 to 8.7, more preferably from 7.2 to 8.2, even more preferably from 7.5 to 7.9, in particular 7.7. The pH is determined in the manner described in the experimental part. In order to stabilize the pH at the range described above, the hydrogel used according to the invention, in an alternative embodiment, can also contain appropriate buffer substances or a buffer solution having the appropriately adjusted pH. A nonlimiting example of a buffer solution would be a 0.1 molar sodium phosphate solution (pH 7.4). It has been found that the aforementioned pH range can allow an advantageous treatment of pressure ulcers and/or the prophylaxis of the development of pressure ulcers.

The matrix of the hydrogel used according to the invention can preferably be composed of a synthetic or natural material, more preferably of a synthetic or natural polymer material. Examples of natural polymers which can be used as matrix for the hydrogel used according to the invention are agar, alginic acid and salts and derivatives thereof, guar gum, chitin and derivatives thereof, chitosan (derivatives), carrageenan, xanthan gum, gum arabic, tragacanth, gellan gum, pectin and mixtures thereof. These polymers can also be considered to be plant polymer material for forming hydrogels. Further examples are gelatin, peptin and glycoproteins and mixtures thereof. These polymers can also be considered to be animal polymer material.

In a further preferred embodiment, cellulose and/or derivatives thereof can also be used as matrix for the hydrogel used according to the invention. In the context of the present invention, the group of cellulose derivatives includes, in particular, cellulose ethers and cellulose esters and also salts thereof. The cellulose ethers used here are preferably hydroxyalkylcelluloses, in particular hydroxy-C₁₋₆-alkylcellulose such as hydroxymethylcellulose, hydroxyethylcellulose, hydroxypropylcellulose or hydroxybutylcellulose and very particularly preferably hydroxymethylcellulose or hydroxyethylcellulose. The cellulose esters used are, in particular, carboxyalkylcellulose, in particular carboxy-C₁₋₆-alkylcellulose such as carboxymethylcellulose, carboxyethylcellulose, carboxypropylcellulose or carboxybutylcellulose or salts thereof and very particularly preferably carboxymethylcellulose or carboxyethylcellulose or salts thereof, in particular sodium salts thereof. Furthermore, mixtures thereof can be used. The number average molecular weight of the celluloses and/or derivatives thereof is preferably 1000-250 000 g/mol, more preferably 5000-175 000 g/mol, in particular 10 000-100 000 g/mol. The number average molecular weight is preferably determined using gel permeation chromatography.

In a further preferred embodiment, the matrix of the hydrogel used according to the invention can also be a synthetic polymer. The synthetic polymer generally has a number average molecular weight of from 2500 to 250 000 000 g/mol, preferably from 5000 to 5 000 000 g/mol, more preferably from 50 000 to 1 000 000 g/mol. Examples of synthetic polymers are polyurethane, polyvinyl alcohol, poly(meth)acrylate and polyvinylpyrrolidone.

In a preferred embodiment, the hydrogel used according to the invention can be a polyurethane-based hydrogel. In other words, the matrix of the hydrogel used according to the invention is based on a polyurethane. Hydrogel matrices comprising a polyurethane-polyurea copolymer are especially suitable in the context of the present invention. Said polyurethane-polyurea copolymer can be formed in particular from a prepolymer having aliphatic diisocyanate groups and a polyethylene oxide-based polyamine. In particular, the polyurethane-polyurea copolymer can be formed from an isophorone diisocyanate-terminated prepolymer, a polyethylene oxide-based polyamine, and water.

Furthermore, it is preferred that the hydrogel used according to the invention can also comprise at least one polyhydric alcohol from the group of dihydric, trihydric, tetrahydric, pentahydric or hexahydric alcohols. In particular, the alcohol can be chosen from the group of glycols, in particular ethylene glycol or propylene glycol, and also sorbitol or glycerol or mixtures thereof. These alcohols are excellent moisturizers. The hydrogel according to the invention can comprise from 0% to 50% by weight of a polyhydric alcohol, more preferably from 5% to 40% by weight of a polyhydric alcohol and very particularly preferably from 10% to 30% by weight of a polyhydric alcohol.

Furthermore, the hydrogel used according to the invention can comprise in particular at least one salt. In particular, the hydrogel matrix comprises here an inorganic salt. Chlorides, iodides, sulfates, hydrogensulfates, carbonates, hydrogencarbonates, phosphates, dihydrogenphosphates or hydrogenphosphates of alkali and alkaline earth metals are particularly suitable in this connection. Very particularly preferably, the hydrogel matrix comprises sodium chloride, potassium chloride, magnesium chloride, calcium chloride or mixtures thereof. These salts are particularly good simulators of the electrolyte mixture in a wound serum released by a wound. Thus, the hydrogel used according to the invention and comprising these salts provides a wound with conditions that are particularly conducive to wound healing. Here, the hydrogel used according to the invention can comprise from 0% to 5% by weight, preferably from 0.1% to 3% by weight, very particularly preferably from 0.5% to 1.5% by weight of at least one salt.

According to a preferred embodiment, the hydrogel used according to the invention is composed of from 6% to 30% by weight of a prepolymer having aliphatic diisocyanate groups, from 4% to 20% by weight of polyethylene oxide-based diamine, from 5% to 30% by weight of a polyhydric alcohol selected from the group consisting of propylene glycol and/or glycerol, 0.5-1.5% by weight of a salt selected from the group consisting of sodium chloride, potassium chloride, magnesium chloride, calcium chloride or mixtures thereof, and from 40% to 80% by weight of water.

More preferably, the hydrogel used according to the invention is composed of from 6% to 20% by weight, in particular about 12.6% by weight of isophorone diisocyanate-terminated prepolymer, from 4% to 15% by weight, in particular about 8.7% by weight of polyethylene oxide-based diamine, from 15% to 20% by weight, in particular about 16.5% by weight/or glycerol, from 0.5% to 1.5% by weight, in particular about 1.0% by weight of a salt, preferably sodium chloride, and from 50% to 80% by weight, in particular about 61.3% by weight of water.

Furthermore, the present invention relates to the use of a hydrogel for production of a wound dressing for treatment of pressure ulcers and/or prophylaxis of the development of pressure ulcers, wherein the hydrogel has a water content of from 40% to 90%.

With regard to the pressure ulcers and the properties of the hydrogel, what was described above applies. For example, the wound dressing produced using the hydrogel is preferably used in the treatment of device-related pressure ulcers, and the hydrogel has a water content of from 40% to 90%, preferably from 60% to 85%. The hydrogel used for production of a wound dressing can preferably be fixed to the affected area of the skin. This can be done solely through the contact pressure of the device, for example protective goggles, on the wound dressing produced using the hydrogel, which wound dressing is arranged between the affected area of the skin and the device itself.

Alternatively, the hydrogel used for production of a wound dressing can be fixed to the affected area of the skin with the aid of a fixative such as an adhesive. Here, for example, the edge region of a layer overlapping the hydrogel and arranged on the side facing away from skin can be provided with a fixative such as an adhesive, so that the hydrogel can be directly applied and fixed to the affected area of the skin. Suitable fixatives such as medically acceptable adhesives are known to a person skilled in the art.

The wound dressing can preferably be in the form of a variant having a frame (“island dressing”). In a preferred embodiment of the invention, the hydrogel used is present in a thickness of from 0.5 to 2.0 mm, preferably from 0.6 to 1.8 mm, in particular from 1.0 to 1.6 mm. This thickness can achieve agreeable wearing comfort and ensure sufficient prevention of pressure peaks due to the device on the skin, thus making it possible to advantageously prevent the development of a pressure ulcer.

As an alternative preference, the wound dressing can be in the form of a variant without a frame. In a preferred embodiment of the invention, the hydrogel used is present in a thickness of from 1.0 to 2.0 mm, preferably from 1.1 to 1.8 mm, in particular from 1.2 to 1.6 mm. This thickness can achieve agreeable wearing comfort and ensure sufficient prevention of pressure peaks due to the device on the skin, thus making it possible to advantageously prevent the development of a pressure ulcer.

The hydrogel used for production of a wound dressing is preferably transparent. In the context of the present invention, transparency is considered to be sufficient permeability of a material, in this case hydrogel, to light in the visible spectrum. Thus, in the case of a transparent material, the side of the material facing away from the viewer's eye can be viewed through the material. In other words, the transparency of the hydrogel makes it possible for the condition of the skin area under the hydrogel to be viewed by inspection and subsequently assessed without the wound dressing produced using the hydrogel having to be removed from the affected skin area. The transparency is thus preferably determined by a visual check. As a result, it is possible, for example, to avoid unnecessary changing of the wound dressing, which firstly spares the patient the resultant pain and secondly avoids an unnecessarily high material expenditure.

The wound dressing produced using the hydrogel preferably comprises a cover layer which is attached to the side of the hydrogel facing away from the skin. Films or foams made from polyurethane, polyester, polyether urethane, polyester urethane, polyether-polyamide copolymers, polyacrylate or polymethacrylate are preferably suitable as cover layer. In particular, a water-impermeable and water vapor-permeable polymer film is suitable as cover layer. In particular, a polyurethane film, polyester urethane film or polyether urethane film is preferred as polymer film. However, polymer films having a thickness of from 15 to 60 μm, in particular from 20 to 40 μm and very particularly preferably from 25 to 30 μm are also very particularly preferred. The cover layer preferably has a moisture vapor transmission rate (MVTR) of from 250 g/m²/24 hours to 1000 g/m²/24 hours, more preferably from 300 g/m²/24 hours to 750 g/m²/24 hours (measured in accordance with DIN EN 13726-2:2002, upright). As an alternative preference, the moisture vapor transmission rate of the cover layer, in particular of the polymer film, is at least 300 g/m²/24 hours, in particular at least 1000 g/m²/24 hours and very particularly preferably at least 2000 g/m²/24 hours up to for example 5000 g/m²/24 hours or 10 000 g/m²/24 hours (measured in accordance with DIN EN 13726-2:2002, upright). In a preferred embodiment, the cover layer can be covered with an adhesive layer. In particularly preferred embodiments, the cover layer has a moisture-proof, adhesive edge section. Said edge section ensures that the hydrogel can be applied and fixed to the affected skin areas.

A further subject of the invention is a kit containing a hydrogel and a protective or artificial respiration mask or protective goggles, wherein the hydrogel has a water content of from 40% to 90%.

With regard to the properties of the hydrogel, what was described above applies.

The hydrogel contained in the kit is preferably in the form of a layered material. The individually required dimensions of the hydrogel for the affected skin areas of the patient can then be cut from this with the aid of a preferably sterile cutting device such as a scalpel or scissors, and applied and, if necessary, fixed before the protective or artificial respiration mask is fitted or protective goggles are fitted. In a preferred embodiment, the kit according to the invention contains a cutting device, in particular scissors. As a result of the application of the hydrogel before the fitting of a protective or artificial respiration mask or protective goggles, the pressure peaks exerted on the skin by the aforementioned device are distinctly reduced, thus advantageously reducing the development of device-related pressure ulcers.

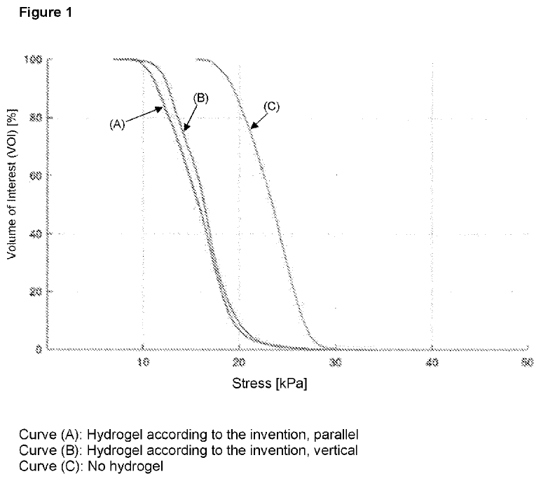

FIG. 1 shows a graph showing the results of the FEM (finite element method) in relation to the hydrogel used according to the invention. The abscissa shows von Mises stresses occurring on the skin. The ordinate shows the proportion of the relevant comparison volume (volume of interest, VOI) having von Mises stresses at least as great as the associated abscissa intercepts.

### EXPERIMENTAL PART

### 1. Methods of Determination

### 1.1 Water Content of the Hydrogel

The method is carried out as follows:
Transfer a weighing bottle and its removed cap to a drying cabinet, heat at 105° C. for 30 minutes, cool in a desiccator for 30 minutes, and finally determine the weight of the weighing bottle and cap (weight W₁).
Weigh about 5 grams of the sample substance into the weighing bottle and determine the weight (weight W₂).
Dry the sample in the drying cabinet at 105° C. for at least 30 minutes until the weight remains constant (difference between two measurements is less than 0.1% of the initial value). Put the cap on and cool in a desiccator for 30 minutes.
Briefly lift the cap for pressure equalization and then reclose the weighing bottle.
Determine the weight of the closed weighing bottle (weight W₃).

The water content uₘ is calculated as follows:

$${u}_{m} [ \% ] = \frac{{W}_{1} + {W}_{2} - {W}_{3}}{{W}_{2}} \times 100$$

W₁: Weight of the weighing bottle with cap
W₂: Weight of the sample
W₃: Weight of the treated sample and the weighing bottle with cap

The method is carried out five times and the water content is determined as the mean of the values obtained in each case.

### 1.2 Water Releasing Ability of the Hydrogel

As a preparatory step, sheets of filter paper of 5.5 centimeters in diameter are conditioned in air at 23° C. and 50±4% relative humidity for 24 hours.

The method is carried out as follows:
Place 5 conditioned sheets of filter paper on top of one another in a Petri dish of 6.5 cm in diameter and determine the weight of the Petri dish with the sheets of filter paper (weight M₀).
Punch out a sample of 5 cm in diameter and place the sample on the sheets of filter paper.
Place a plastic disk of 5.5 cm in diameter and of 10±0.3 g in weight on the sample.
Close the Petri dish with a lid and seal it with a plastic film such as Parafilm.
Store at 37° C. for 24 hours.
Remove the plastic film, the lid, the plastic disk and the sample.
Determine the weight of the Petri dish with the sheets of filter paper (weight Mₜ).

The water releasing ability is calculated as follows:

$$M {D}_{t} = \frac{( {M}_{t} - {M}_{0} ) \times 1000}{A}$$

M₀: Weight of the Petri dish with the sheets of filter paper (in g) at time t=0
Mₜ: Weight of the Petri dish with the sheets of filter paper (in g) at time t=24 hours
A: Area of the sample in cm²

### 1.3 Absorption Capacity of the Hydrogel

The method is carried out as follows:
Punch out a 50×50 mm sample.
Determine the weight of the sample (M₁).
Place the sample in a beaker of demineralized water for 24 hours.
Remove the sample from the beaker and determine the weight of the sample (M₂).

The adsorption capacity WA is calculated as follows:

$$WA [ g / g ] = \frac{{M}_{2} - {M}_{1}}{{M}_{1}}$$

M₁: Weight of the sample before absorption
M₂: Weight of the sample after absorption for 24 hours

### 1.4 Determination of pH

Instrument: pH meter with combination electrode in accordance with DIN 19623:2007-05, suitable for measuring at least 0.05 pH units

The method is carried out as follows:
Cut an approximately 2.5×2.5 cm sample and determine the sample weight.
Transfer the sample to a beaker and add five times the sample weight of demineralized water.
Cover the beaker with a watch glass and leave for 24 hours at 23° C.
Remove the sample and immerse the electrode in the liquid that remains for two minutes.
Read the pH on the display.

Production of a Polyurethane-Based Hydrogel

A hydrogel used according to the invention can be produced by mixing (reacting) a mixture of components 1) to 4) with component 5) and then transferring the mixture to a mold in order to obtain the desired thickness.

| | |
|---|---|
| 1) Aqua Purificata | 61.31% by weight |
| 2) Sodium chloride | 0.96% by weight |
| 3) Glycerol Ph. Eur. (99.7%) | 16.48% by weight |
| 4) Isocyanide, e.g., Aquapol PI-1300-31 | 12.58% by weight |
| 5) Diamine, e.g., Jeffamine ED-2003 | 8.67% by weight |

Finite Element Method (FEM)

The FEM is a general and computer-aided numerical method used for different physical problems.

The FEM is logically based on numerically solving a complex system of differential equations.

The FEM divides large problems into a multitude of smaller parts called finite elements. Analysis is carried out with each of these elements and, taken as a whole, results in a solution for the entire problem.

The work steps of an FEM can be described as follows:
1. creation of a 2D or 3D model consisting of finite elements;
2. definition of the material properties of the model;
3. definition of the boundary conditions and loads for application of the model to the problem;
4. computer-aided solving of the problem; and
5. analysis of the results through visualization and calculation.

The FEM calculation underlying the invention was done according to the method described in the following article: Levy A, Schwartz D, Gefen A. The contribution of a directional preference of stiffness to the efficacy of prophylactic sacral dressings in protecting healthy and diabetic tissues from pressure injury: computational modelling studies. Int. Wound J 2017; doi: 10.111/iwj 0.12821

To understand the effects of the hydrogel used according to the invention, FE models of a human pelvis and a hydrogel layer were created. The influences of pressure and stress on the skin and deeper tissues were analyzed.

The pelvic model was based on MRI scans of a female test subject in order to ensure 20 the greatest possible anatomical accuracy of the model. The FE models comprise 3 900 000 nodes. Soft tissues were represented as nonlinear materials, with muscles being amalgamated to form one material and fat and skin each being amalgamated as compressible materials. The bones were amalgamated as a rigid body.
Modeling was based on the following material properties:
Bone: Linear modulus of elasticity E=7000 MPa
Adipose tissue: Hyperelasticity (neo-Hooke) C10=0.0004
Muscle tissue: Hyperelasticity (neo-Hooke) C10=0.000225
Skin: Hyperelasticity (neo-Hooke) C10=0.004

A relevant model volume (volume of interest, VOI) having dimensions of 6.7 cm×2.0 cm×5.1 cm (x-direction×y-direction×z-direction) was formed, containing the sacrum (os sacrum) and the surrounding soft tissue including skin.

Von Mises stress refers to a fictitious uniaxial stress which, on the basis of a specific material-mechanical or mathematical criterion, represents a hypothetically equivalent material stress, such as a real multiaxial state of stress. On the basis of the von Mises stress, the real, generally three-dimensional state of stress in the component in the strength or yield condition can be compared with the characteristics from the uniaxial tensile test (material characteristics, for example yield point or tensile strength).

The von Mises stresses can be calculated according to the following formula:

$${σ}_{v , M} = \sqrt{\frac{1}{2} [ {( {σ}_{I} - {σ}_{II} )}^{2} + {( {σ}_{II} - {σ}_{III} )}^{2} + {( {σ}_{III} - {σ}_{I} )}^{2} ]}$$

Here, GI, GIL and GM are the principal stresses occurring in the three spatial directions.

What are of particular importance are the stresses occurring on the skin within the relevant model volume, because what occur here are not only the pressures responsible for the development and worsening of decubitus ulcers, but also shearing forces.

A comparison was made in each case between calculations in which a hydrogel used according to the invention having a thickness (including a cover layer) of 1.4 mm was placed on a skin area with the same skin area without an applied hydrogel.

What is of significant interest is the 10% value. This value indicates what maximum stresses occur in not more than 10% of the relevant comparison volume. It corresponds to the curve point associated with the ordinate intercept at 10% VOI.

From the comparison of the von Mises stresses on the skin in the relevant model volume with and without an applied hydrogel used according to the invention, suitability for prevention of the development of pressure ulcers such as device-related pressure ulcers can be postulated if the 10% value for the stresses that occur is reduced by more than 10%.

The curves shown in FIG. 1 have the following 10% values:
Curve (A): Hydrogel according to the invention, parallel: 19.0 kPa
Curve (B): Hydrogel according to the invention, vertical: 20.0 kPa
Curve (C): No hydrogel: 26.5 kPa

As can be seen from FIG. 1, the 10% value for the von Mises stresses that occur on the skin in the relevant model volume was reduced by over 20% by use of a hydrogel used according to the invention.

1-15. (canceled)

16. A hydrogel for use in the treatment of pressure ulcers and/or prophylaxis of the development of pressure ulcers, wherein the hydrogel has a water content of from 40% to 90%.

17. The hydrogel of claim 16, wherein the pressure ulcers are device-related pressure ulcers.

18. The hydrogel of claim 16, wherein the hydrogel has a water content of from 60% to 85%.

19. The hydrogel of claim 16, wherein the hydrogel can release 10±2 mg of moisture per square centimeter (cm²) within 24 hours.

20. The hydrogel of claim 16, wherein the hydrogel has an absorption capacity of at least 1 g/g (hydrogel) within 24 hours.

21. The hydrogel of claim 16, wherein the hydrogel has a pH of from 5 to 9.

22. The hydrogel of claim 16, wherein the hydrogel is a polyurethane-based hydrogel.

23. A method of treating a pressure ulcer and/or preventing the development of a pressure ulcer comprising applying a wound dressing to an affected area of skin of a patient, wherein the wound dressing comprises a hydrogel having a water content from 40% to 90%.

24. The method of claim 23, wherein the hydrogel is fixed to the affected area of the skin.

25. The method of claim 23, wherein the hydrogel is present in a thickness of from 0.5 mm to 2.0 mm.

26. The method of claim 23, wherein the hydrogel is present in a thickness of from 1.0 mm to 2.0 mm.

27. The method of claim 23, wherein the hydrogel is transparent.

28. The method of claim 23, wherein the wound dressing comprises a cover layer which is attached to the side of the hydrogel facing away from the skin.

29. The method of claim 28, wherein the cover layer has a moisture vapor transmission rate (MVTR) of from 250 g/m²/24 hours to 1000 g/m²/24 hours.

30. The method of claim 23, wherein the hydrogel can release 10±2 mg of moisture per square centimeter (cm²) within 24 hours.

31. The method of claim 23, wherein the affected area of the skin is one or more contact points between the skin and a medical device.

32. The method of claim 31, wherein the medical device is a protective or artificial respiration mask or protective goggles.

33. The method of claim 23, wherein the hydrogel has an absorption capacity of at least 1 g/g (hydrogel) within 24 hours.

34. The method of claim 23, wherein the hydrogel is a polyurethane-based hydrogel and has a pH of from 5 to 9.

35. A kit containing a hydrogel as claimed in claim 16 and a protective or artificial respiration mask or protective goggles, wherein the hydrogel has a water content of from 40% to 90%.
